(19) United States
(12) Patent Application Publication
(10) Pub. No.: US 2021/0177870 A1
(43) Pub. Date: Jun. 17, 2021

(54) COMPOSITION CONTAINING PLASMALOGEN FOR ENHANCING MEMORIZATION ABILITY

(71) Applicants: NIHON PHARMACEUTICAL CO., LTD., Tokyo (JP); SUNSHO PHARMACEUTICAL CO., LTD., Fuji-shi, Shizuoka (JP)

(72) Inventors: Tatsuya WADA, Tokyo (JP); Yoshiharu MATAHIRA, Fuji-shi (JP); Hirofumi WATANABE, Fuji-shi (JP); Masaki OOKAWARA, Fuji-shi (JP)

(73) Assignees: NIHON PHARMACEUTICAL CO., LTD., Tokyo (JP); SUNSHO PHARMACEUTICAL CO., LTD., Fuji-shi, Shizuoka (JP)

(21) Appl. No.: 17/270,609

(22) PCT Filed: Aug. 23, 2019
(86) PCT No.: PCT/JP2019/033038

§ 371 Date: Feb. 23, 2021

(30) Foreign Application Priority Data
Aug. 24, 2018 (JP) 2018-157356

(51) Int. Cl. A61K 31/685; A61P 25/28

(52) U.S. Cl. CPC A61K 31/685; A61P 25/28

(57) ABSTRACT

This is to provide a useful application of plasmalogen, which is not yet known.

A composition contains plasmalogen for enhancing a verbal memorization ability and/or a visual memorization ability.

### TECHNICAL HELD

The present invention relates to a composition containing plasmalogen for enhancing memorization ability. More specifically, the present invention relates to a composition containing plasmalogen for enhancing memorization power such as a verbal memorization ability, a visual memorization ability, a total memorization ability, and the like.

### BACKGROUND ART

There have been reported with a large number that plasmalogen participates in signal transduction in the brain, has a function as an antioxidant in the brain, and the like. For example, in Patent Document 1, it has been reported that plasmalogen has an effect of brain nerve cell neoplasia, and in Patent Document 2, it has been reported that plasmalogen has an effect of improving central nervous system inflammation.

Recently, it has also been reported that a learning memorization ability was enhanced in mice to which plasmalogen was administered (Patent Document 3).

### PRIOR ART DOCUMENTS

### Patent Documents

Patent Document 1: WO 2011/083827A

Patent Document 2: WO 2012/039472A

Patent Document 3: JP 2016-210696A

### SUMMARY OF THE INVENTION

### Problems to be Solved by the Invention

As mentioned above, there have been reported that plasmalogen has a wide range of functions in vivo, such as an effect of improving central nervous system inflammation, an effect of enhancing a learning memorization ability, and the like. However, it has been considered that the entire functions of plasmalogen in vivo have not yet been elucidated. Therefore, there is a possibility that plasmalogen may have other beneficial functions that are not yet known.

### Means to Solve the Problems

The present inventors have intensively studied in view of the above-mentioned tasks, and as a result, they have found that plasmalogen has surprisingly a function of enhancing a verbal memorization ability and/or a visual memorization ability, whereby the present invention has been completed.

Accordingly, the present invention provides, as a gist, the following.

[1] A composition containing plasmalogen for enhancing a verbal memorization ability and/or a visual memorization ability.
[2] The composition described in [1], for enhancing a verbal memorization ability.
[3] The composition described in [1] or [2], for enhancing a verbal memorization ability and a visual memorization ability.
[4] The composition described in any of [1] to [3], wherein the plasmalogen comprises ethanolamine plasmalogen and choline plasmalogen.
[5] The composition described in [4], wherein a mass ratio of ethanolamine plasmalogen to choline plasmalogen comprised in the plasmalogen is 7:3 to 9:1.
[6] The composition described in any of [1] to [5], wherein the plasmalogen is derived from a plasmalogen-containing animal tissue.
[7] The composition described in [6], wherein the animal tissue is derived from aquatic invertebrates.
[8] The composition described in [7], wherein the aquatic invertebrates are aquatic invertebrates of phylum Chordata, subphylum Urochordata, class Ascidiacea.
[9] The composition described in [8], wherein the aquatic invertebrates of the phylum Chordata, subphylum Urochordata, class Ascidiacea are aquatic invertebrates of the genus Halocynthia.
[10] The composition described in [9], wherein the aquatic invertebrates of the genus Halocynthia is ascidian or golden sea squirt.
[11] The composition described in any of [1] to [10], for enhancing a healthy verbal memorization ability.
[12] The composition described in any of [1] to [11], wherein 12 to 25 μg/kg body weight per day of plasmalogen is administered.
[13] The composition described in any of [1] to [12], which is a food composition.
[14] The composition described in [13], wherein the food composition is a food for specified health uses or a food with functional use, or a health food other than these.

### Effects of the Invention

According to the present invention, it is possible to enhance a verbal memorization ability and/or a visual memorization ability in a subject who has ingested plasmalogen.

### BRIEF DESCRIPTION OF THE DRAWINGS

FIG. 1 shows the result of a verbal memorization ability in a human clinical test carried out in Test Example 1. Incidentally, “*” indicates p<0.1.

FIG. 2 shows the result of a visual memorization ability in a human clinical test carried out in Test Example 1. Incidentally, “**” indicates p<0.05.

FIG. 3 shows the result of a total memorization ability in a human clinical test carried out in Test Example 1. Incidentally, “**” indicates p<0.05.

FIG. 4A shows the result (graph) of analyzing a ratio of ethanolamine plasmalogen comprised in plasmalogen by LCMSMS.

FIG. 4B shows the result (table) of analyzing a ratio of ethanolamine plasmalogen comprised in plasmalogen by LCMSMS.

FIG. 5A shows the result (graph) of analyzing a ratio of choline plasmalogen comprised in plasmalogen by LCMSMS.

FIG. 5B shows the result (table) of analyzing a ratio of choline plasmalogen comprised in plasmalogen by LCMSMS.

### EMBODIMENTS TO CARRY OUT THE INVENTION

In one embodiment of the present invention, a composition containing plasmalogen for enhancing a verbal memorization ability and/or a visual memorization ability is provided.

The “verbal memorization ability” used in the present specification is an ability to recognize, memorize, and recall afterward characters, words, and so on, and means a processing ability of expressions and meanings of characters, words, and so on. The verbal memorization ability can be evaluated, for example, by a language memory test using Cognitrax.

The “visual memorization ability” used in the present specification is an ability to recognize, memorize, and recall afterward figures, spaces, and so on, and means a processing ability of figures and spatial expressions. The visual memorization ability can be evaluated, for example, by a visual memory test using Cognitrax.

In another embodiment of the present invention, it is provided a method for enhancing a verbal memorization ability and/or a visual memorization ability, comprising administering an effective amount of plasmalogen to a subject in need thereof.

In another embodiment of the present invention, it is provided plasmalogen for enhancing a verbal memorization ability and/or a visual memorization ability.

In another embodiment of the present invention, it is provided use of plasmalogen for producing foods and pharmaceuticals for enhancing a verbal memorization ability and/or a visual memorization ability.

In one embodiment of the present invention, it is provided a composition containing plasmalogen for enhancing a verbal memorization ability.

In one embodiment of the present invention, it is provided a composition containing plasmalogen for enhancing a verbal memorization ability and a visual memorization ability.

The “total memorization ability” used in the present specification means, for example, when it is measured by using Cognitrax, a memorization ability calculated from the sum of a verbal memorization test and a visual memorization test. Accordingly, in another embodiment of the present invention, it is provided a composition containing plasmalogen for enhancing a total memorization ability.

The “plasmalogen” used in the present specification means, in general, a glycerophospholipid having a long-chain alkenyl group through a vinyl ether bond at the 1-position (sn-1 position) of the glycerol skeleton, and, for example, can be represented by the following general formula:

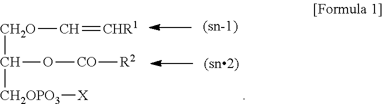

R¹ is an aliphatic hydrocarbon group and is generally an aliphatic hydrocarbon group having 1 to 20 carbon atoms. As R¹, which is not limited to these, there may be mentioned, for example, a dodecyl group (C12), a tetradecyl group (C14), a hexadecyl group (C16), an octadecyl group (C18), an octadecenyl group (C18:1), an eicosanyl group (C20) (preferably a hexadecyl group (C16), an octadecyl group (C18), an eicosanyl group (C20), more preferably an octadecyl group (C18)), and the like.

R² is an aliphatic hydrocarbon group and is generally a group corresponding to an aliphatic hydrocarbon portion of a fatty acid. As R², which is not limited to these, there may be mentioned, for example, a group corresponding to an aliphatic hydrocarbon portion of a fatty acid such as palmitic acid, stearic acid, oleic acid, octadecadienoic acid, octadecatrienoic acid, eicosenoic acid, eicosatetraenoic acid, eicosapentaenoic acid, docosatetraenoic acid, docosapentaenoic acid, docosahexaenoic acid, and the like. More specifically, R²—CO— may be mentioned, for example, a group derived from palmitic acid (CH₃(CH₂)₁₄—CO—), a group derived from stearic acid (CH₃(CH₂)₁₆—CO—), a group derived from oleic acid (CH₃(CH₂)₇CH═CH(CH₂)₇—CO—), a group derived from octadecadienoic acid (for example, CH₃(CH₂)₄(CH═CHCH₂)₂(CH₂)₆—CO—), a group derived from octadecatrienoic acid (for example, CH₃CH₂(CH═CHCH₂)₃(CH₂)₆—CO—), a group derived from eicosenoic acid (CH₃(CH₂)₇CH═CHCH₂(CH₂)₈—CO—), a group derived from eicosatetraenoic acid (for example, CH₃(CH₂)₄(CH═CHCH₂)₄(CH₂)₂—CO—), a group derived from eicosapentaenoic acid (for example, CH₃CH₂(CH═CHCH₂)₅(CH₂)₂—CO—), a group derived from docosatetraenoic acid (for example, CH₃(CH₂)₄(CH═CHCH₂)₄(CH₂)₄—CO—), a group derived from docosapentaenoic acid (for example, CH₃CH₂(CH═CHCH₂)₅(CH₂)₄—CO—, CH₃(CH₂)₄ (for example, CH═CHCH₂)₅CH₂—CO—), a group derived from docosahexaenoic acid (for example, CH₃CH₂(CH═CHCH₂)₆CH₂—CO—), and the like.

X represents a polar group, and there may be mentioned, which is not limited to these, for example, —CH₂CH₂N⁺H₃, —CH₂CH₂N⁺(CH₃)₃ and —CH₂CH(NH₂)COOH (preferably —CH₂CH₂N⁺H₃ and —CH₂CH₂N⁺(CH₃)₃), and the like.

In the above-mentioned general formula, in the case where X is —CH₂CH₂N⁺H₃, it shows ethanolamine plasmalogen, and in the case where X is —CH₂CH₂N⁺(CH₃)₃, it shows choline plasmalogen. It is preferable that the composition described in the present specification comprises ethanolamine plasmalogen and choline plasmalogen as the plasmalogen.

Accordingly, in one embodiment of the present invention, it is provided a composition described in the present specification in which the plasmalogen comprises ethanolamine plasmalogen and choline plasmalogen.

In another embodiment of the present invention, it is provided a method for enhancing a verbal memorization ability and/or a visual memorization ability described in the present specification in which plasmalogen comprises ethanolamine plasmalogen and choline plasmalogen.

In another embodiment of the present invention, it is provided plasmalogen for enhancing a verbal memorization ability and/or a visual memorization ability described in the present specification in which the plasmalogen comprises ethanolamine plasmalogen and choline plasmalogen.

A mass ratio of ethanolamine plasmalogen to choline plasmalogen comprised in the plasmalogen is not particularly limited as long as the objects of the present invention are accomplished and, for example, it is about 5:about 5 to about 10:about 0, preferably about 6:about 4 to about 9.5:about 0.5, more preferably about 7:about 3 to about 9:about 1, and further preferably about 8:about 2 to about 8.8:about 1.2.

In a preferred embodiment of the present invention, the composition described in the present specification in which a mass ratio of ethanolamine plasmalogen to choline plasmalogen comprised in the plasmalogen is about 7:about 3 to about 9:about 1 is provided.

In another embodiment of the present invention, it is provided a method for enhancing a verbal memorization ability and/or a visual memorization ability described in the present specification in which a mass ratio of ethanolamine plasmalogen to choline plasmalogen comprised in the plasmalogen is about 7:about 3 to about 9:about 1.

In another embodiment of the present invention, it is provided plasmalogen for enhancing a verbal memorization ability and/or a visual memorization ability described in the present specification in which a mass ratio of ethanolamine plasmalogen to choline plasmalogen comprised in the plasmalogen is about 7:about 3 to about 9:about 1.

In a more preferred embodiment of the present invention, it is provided a composition described in the present specification in which a mass ratio of ethanolamine plasmalogen to choline plasmalogen comprised in the plasmalogen is about 8:about 2 to about 8.8:about 1.2.

In one embodiment of the present invention, it includes a material in which ethanolamine plasmalogen comprised in the plasmalogen has an octadecyl group at the sn-1 position (that is, R¹ in the above-mentioned general formula is an octadecyl group).

In one embodiment of the present invention, it includes a material in which choline plasmalogen comprised in the plasmalogen has an octadecyl group at the sn-1 position (that is, R¹ in the above-mentioned general formula is an octadecyl group).

In a preferred embodiment of the present invention, it includes a material in which ethanolamine plasmalogen comprised in the plasmalogen has an octadecyl group at the sn-1 position (that is, R¹ in the above-mentioned general formula is an octadecyl group), and also includes a material in which choline plasmalogen comprised in the plasmalogen has an octadecyl group at the sn-1 position (that is, R¹ in the above-mentioned general formula is an octadecyl group).

In one embodiment of the present invention, it includes at least one or more of a material(s) in which ethanolamine plasmalogen comprised in the plasmalogen has a group derived from oleic acid, a group derived from octadecadienoic acid, a group derived from eicosenoic acid, a group derived from eicosatetraenoic acid, a group derived from eicosapentaenoic acid or a group derived from docosahexaenoic acid (preferably a group derived from oleic acid, a group derived from eicosatetraenoic acid, a group derived from eicosapentaenoic acid or a group derived from docosahexaenoic acid) at the sn-2 position (that is, as R²—CO— in the above-mentioned general formula).

In one embodiment of the present invention, it includes at least one or more of a material(s) in which choline plasmalogen comprised in the plasmalogen has a group derived from palmitic acid, a group derived from octadecatrienoic acid, a group derived from eicosapentaenoic acid or a group derived from docosapentaenoic acid at the sn-2 position.

In a preferred embodiment of the present invention, it includes at least one or more of a material(s) in which ethanolamine plasmalogen comprised in the plasmalogen has a group derived from oleic acid, a group derived from octadecadienoic acid, a group derived from eicosenoic acid, a group derived from eicosatetraenoic acid, a group derived from eicosapentaenoic acid or a group derived from docosahexaenoic acid (preferably a group derived from oleic acid, a group derived from eicosatetraenoic acid, a group derived from eicosapentaenoic acid or a group derived from docosahexaenoic acid) at the sn-2 position, and includes at least one or more of a material(s) in which choline plasmalogen comprised in the plasmalogen has a group derived from palmitic acid, a group derived from octadecatrienoic acid, a group derived from eicosapentaenoic acid or a group derived from docosapentaenoic acid at the sn-2 position.

The plasmalogen (or a composition containing plasmalogen) described in the present specification may be, which is not limited to these, for example, plasmalogen (or a composition containing plasmalogen) synthesized by a known or well-known method, or may be plasmalogen (or a composition containing plasmalogen) obtained from a living tissue by a known or well-known method or by the method mentioned later and the like. As the living tissue, which is not limited to these, there may be mentioned, for example, a plasmalogen-containing animal tissue, a plasmalogen-containing microorganism tissue and the like, and preferably a plasmalogen-containing animal tissue.

The “plasmalogen-containing animal tissue” used in the present specification is not particularly limited as long as it is an animal tissue containing plasmalogen, and may be an entire animal individual, may be a tissue isolated from an animal such as muscle tissue, adipose tissue, nerve tissue, visceral tissue, skin tissue, egg, outer shell, blood, etc., of the animal, or may be a combination of an entire animal individual and an isolated tissue or a mixture of a plurality of isolated tissues. As the plasmalogen-containing animal tissue, there may be mentioned materials derived from, which is not limited to these, for example, land vertebrates (provided that human is excluded) such as cattle, pig, horse, sheep, goat, chicken, duck, etc.; aquatic vertebrates such as bluefin tuna, salmon, saury, bonito, sardine, cod, etc.; and aquatic invertebrates such as ascidian, golden sea squirt, Asterias amurensis, Asteria pectinifera, Strongylocentrotus nudus, Hemicentrotus pulcherrimus, sea cucumber, Anthopleura fuscoviridis Carlgren, Anthopleura uchidai, scallop, Acanthopleura japonica, Reishia bronni, Nucella freycineti, Chlorostoma lischkei, Tugali gigas, Mytilus galloprovincialis, Septifer virgatus, Crassostrea gigas, octopus, squid, crab, shrimp, etc., these may be used whole individuals or may be used an isolated tissue, and these may be used one kind alone or may be used in combination of two or more kinds.

In a preferred embodiment of the present invention, the animal tissue is derived from aquatic invertebrates and is more preferably aquatic invertebrates of phylum Chordata, subphylum Urochordata.

As the aquatic invertebrates of the phylum Chordata, subphylum Urochordata, there may be mentioned, for example, aquatic invertebrates such as phylum Chordata, subphylum Urochordata, class Ascidiacea; phylum Chordata, subphylum Urochordata, class Thaliacea; phylum Chordata, subphylum Urochordata, class Appendiculata; etc.

In a further preferred embodiment of the present invention, the above-mentioned aquatic invertebrates are aquatic invertebrates of the phylum Chordata, subphylum Urochordata, class Ascidiacea.

In an even more preferred embodiment of the present invention, the above-mentioned aquatic invertebrates of the phylum Chordata, subphylum Urochordata, class Ascidiacea are aquatic invertebrates of the genus Halocynthia. Preferred aquatic invertebrates of the genus Halocynthia are ascidian or golden sea squirt.

The “plasmalogen-containing microorganism tissue” used in the present specification is not particularly limited as long as it is a microorganism tissue containing plasmalogen, and may be a microorganism itself or may be a material isolated from a microorganism such as cell membrane, etc. As the plasmalogen-containing microorganism tissue, which is not limited to these, there may be mentioned, for example, a bacterium of the family Acidaminococcaceae; a rumen bacterium; microorganisms belonging to the genus Propionibacterium such as Propionibacterium acidipropionici (P. acidipropionici), Propionibacterium acnes (P. acnes) and the like.

When the plasmalogen (or a composition containing plasmalogen) is obtained from the plasmalogen-containing animal tissue, the plasmalogen (or a composition containing plasmalogen) can be obtained by the method of, for example, (A) concentrating an alcohol extract of a plasmalogen-containing animal tissue, and (B) after diluting a concentrate obtained in the above-mentioned (A), allowing to stand under refrigeration.

The “alcohol extract of the plasmalogen-containing animal tissue” in the above-mentioned (A) is not particularly limited as long as it is a liquid in which the plasmalogen-containing animal tissue is extracted with a solvent containing an alcohol.

As the “solvent containing an alcohol” may be a solvent comprising an alcohol alone, or may be a mixed solvent of an alcohol and the other solvent(s).

The “alcohol” to be used for extraction, which is not limited to these, may be mentioned, for example, a primary alcohol such as methanol, ethanol, propanol, 1-butanol, etc.; a secondary alcohol such as isopropanol, 2-butanol, etc.; a tertiary alcohol such as tert-butanol, etc., these may be used alone or may be used in combination of two or more kinds.

As the “other solvent(s)” to be used in combination with the alcohol, which is not limited to these, there may be mentioned, for example, water (for example, there may be mentioned common water, natural water, tap water, hard water, soft water, ion-exchanged water, purified water, sterilized water, water for injection, etc., and these may be used alone or may be used in combination of two or more kinds); a fatty acid such as acetic acid, propanoic acid, butanoic acid, pentanoic acid, hexanoic acid, heptanoic acid, octanoic acid, nonanoic acid, oleic acid, etc., or an ester thereof; and a hydrophilic organic solvent such as acetone, etc., other than the above; a hydrophobic organic solvent such as chloroform, hexane, heptane, cyclohexane, petroleum ether, etc.; and the like, and these may be used alone, or may be used in combination of two or more kinds.

When a mixed solvent of the alcohol and the other solvent(s) is used, a mixing ratio of the alcohol and the other solvent(s) is not particularly limited. For example, the ratio of the alcohol:the other solvent(s) is, in volume %, generally about 99.9:about 0.1 to about 0.1:about 99.9, preferably about 99:about 1 to about 50:about 50, more preferably about 99:about 1 to about 60:about 40, and further more preferably about 99:about 1 to about 90:about 10.

An amount of the solvent containing an alcohol to be used for obtaining an extract is not particularly limited, and, for example, it is preferably about 1 to about 100 L per 1 kg of the plasmalogen-containing animal tissue, more preferably about 3 to about 50 L, further preferably about 5 to about 20 L, and even more preferably about 5 to about 10 L.

The method for obtaining the “alcohol extract of the plasmalogen-containing animal tissue” in the above-mentioned (A) is not particularly limited, and, for example, it can be obtained as described below.

The plasmalogen-containing animal tissue is subjected to the extraction treatment in a solvent containing an alcohol at about 1 to about 50° C., preferably about 20 to about 50° C., more preferably about 40 to about 50° C., for about 0.5 to about 24 hours, preferably about 1 to about 10 hours, and more preferably about 2 to about 6 hours, by allowing to stand, mixing or stirring, etc. The solid and liquid are separated into a solid phase 1 and a liquid phase 1 using a strainer, etc. By filtering the liquid phase 1 by a method such as suction filtration, etc., the solid material is removed to obtain a liquid phase 2, and this can be used as an alcohol extract.

Or else, in order to heighten extraction efficiency, a solvent containing an alcohol is added to the separated solid phase 1 as mentioned above, and after dipping at about 1 to about 50° C., preferably about 20 to about 50° C., more preferably about 40 to about 50° C., for about 0.5 to about 24 hours, preferably about 1 to about 10 hours, more preferably about 2 to about 6 hours, the solid and liquid are separated into a solid phase 2 and a liquid phase 3 using a strainer, etc. By filtering the liquid phase 3 by a method such as suction filtration, etc., the solid material is removed to obtain a liquid phase 4. The material in which the liquid phase 2 and the liquid phase 4 are combined can be used as an alcohol extract.

Or else, the material, in which one or more liquid phases obtained by further repeating re-extraction from the solid phase using a solvent containing an alcohol, and the above-mentioned liquid phases 2 and 4 are combined, can be used as an alcohol extract.

Concentration of the alcohol extract in the above-mentioned (A) is not particularly limited and may be carried out in an open system or a closed system, and it is preferably carried out in a closed system. This concentration can be carried out by, for example, concentration by reduced pressure, concentration by heating, concentration by freezing, concentration using a membrane, etc., and among these, concentration by reduced pressure is preferable. Also, this concentration is preferably carried out under bubbling with an inert gas such as nitrogen, argon, etc., in order to prevent oxidation. When concentration by reduced pressure is carried out, it is carried out generally at a temperature of, for example, about 10 to about 55° C., preferably about 25 to about 50° C., more preferably about 40 to about 50° C., and generally for about 1 to about 72 hours, preferably about 6 to about 48 hours, and more preferably about 12 to about 36 hours. As a degree of pressure reduction, there may be mentioned, for example, about 60 mmHg.

In the above-mentioned (B), the solvent to be used for dilution of the concentrate obtained in the above-mentioned (A) is not particularly limited, and, for example, there may be mentioned water (for example, there may be mentioned common water, natural water, tap water, hard water, soft water, ion-exchanged water, purified water, sterilized water, water for injection, etc., and these may be used alone or may be used in combination of two or more kinds); an alcohol such as methanol, ethanol, propanol, 1-butanol, isopropanol, 2-butanol, tert-butanol, etc.; a fatty acid such as acetic acid, propanoic acid, butanoic acid, pentanoic acid, hexanoic acid, heptanoic acid, octanoic acid, nonanoic acid, oleic acid, etc., or an ester thereof; a hydrophilic organic solvent other than the above such as acetone, etc.; a hydrophobic organic solvent such as chloroform, hexane, heptane, cyclohexane, petroleum ether, etc., and these may be used alone, or may be used in combination of two or more kinds. Among these, water or an alcohol is preferable, water or ethanol is more preferable, and water is further more preferable.

When water is used for dilution, pH of water may be adjusted using a pH adjusting agent, if necessary.

As the pH adjusting agent to be used for adjustment of pH of water, which is not limited to these, there may be mentioned, for example, acetic acid, lactic acid, tartaric acid, oxalic acid, glycolic acid, malic acid, citric acid, succinic acid, fumaric acid, phosphoric acid, hydrochloric acid, oxalic acid, sulfuric acid, nitric acid, and a salt thereof, sodium hydroxide, potassium hydroxide, sodium hydrogen carbonate, potassium carbonate, etc., and these may be used alone or may be used in combination of two or more kinds.

An amount of the solvent to be used at the time of dilution is not particularly limited, and, for example, it is preferably about 1 to about 100 L per 1 kg of the concentrate obtained in the above-mentioned (A), more preferably about 10 to about 80 L, and even more preferably about 20 to about 60 L.

Allowing to stand under refrigeration in the above-mentioned (B) is preferably carried out at about 2 to about 15° C., more preferably carried out at about 2 to about 10° C., and further more preferably carried out at about 2 to about 5° C.

Allowing to stand under refrigeration in the above-mentioned (B) is preferably carried out for about 1 to about 7 days, more preferably carried out for about 2 to about 6 days, further more preferably carried out for about 3 to about 5 days, and even further more preferably carried out for about 3 days.

The concentrate obtained in the above-mentioned (A) may be once allowed to stand or stored before diluting it in the above-mentioned (B). The standing or storage herein may be carried out at about 2 to about 15° C., preferably about 2 to about 10° C., more preferably about 2 to about 5° C., and for about 1 to about 14 days, which may be carried out in an open system or may be carried out in a closed system, and it is preferable to carry out in a closed system. Also, the standing or storage herein is preferably carried out under bubbling with an inert gas such as nitrogen, argon, etc., in order to prevent oxidation.

In addition, before diluting the concentrate obtained in the above-mentioned (A) in the above-mentioned (B), if necessary, a material in which the concentrate obtained in the above-mentioned (A) is purified by a method such as solvent extraction, etc., and further concentrated by a method such as filtration under reduced pressure, etc., may be used as a “concentrate” in the above-mentioned (B).

As the above-mentioned plasmalogen-containing animal tissue, a dried material may be used. The drying herein is not particularly limited, and, for example, can be used a known or well-known method such as drying by applying wind, drying by dehumidification, vacuum drying, freeze drying, etc.

As the above-mentioned drying by applying wind, drying by applying wind of generally about 25 to about 59° C., preferably about 35 to about 55° C., and more preferably about 40 to about 50° C. to the plasmalogen-containing animal tissue is preferable. The above-mentioned drying by applying wind may be carried out in an open system or may be carried out in a closed system, and in the case of the open system, an ambient temperature is, for example, about 20 to about 55° C., preferably about 30 to about 45° C., and more preferably about 35 to about 40° C., and in the case of the closed system, an ambient temperature is, for example, preferably substantially the same as the temperature of the wind to be applied to the plasmalogen-containing animal tissue.

The above-mentioned drying by applying wind can be carried out, for example, for about 0.5 to about 96 hours, preferably about 1 to about 72 hours, more preferably about 6 to about 48 hours, and further more preferably about 12 to about 36 hours.

The water content of the dried plasmalogen-containing animal tissue can be, for example, about 1 to about 40% by mass, preferably about 5 to about 30% by mass, and more preferably about 10 to about 25% by mass, based on the whole amount of the plasmalogen-containing animal tissue.

The plasmalogen-containing animal tissue may be divided into two or more parts in order to enhance the efficiency of the above-mentioned drying or the efficiency of alcohol extraction.

After the treatment of the above-mentioned (B), for example, by removing unnecessary fraction(s) by subjecting to a decanting treatment at about 2 to about 15° C., only a fraction containing plasmalogen derived from plasmalogen-containing animal tissue can be obtained. The fraction containing the plasmalogen may be an upper layer, a lower layer, or an intermediate layer other than the above, depending on the kind of the solvent to be used at the time of dilution. In a preferable embodiment, the fraction containing the plasmalogen becomes a lower layer (more preferably the liquid phase which is in the lower layer).

The composition obtained after the treatment of the above-mentioned (B) may be used as such as plasmalogen (or a composition containing plasmalogen) described in the present specification, a material from which unnecessary fractions are removed may be used as plasmalogen (or a composition containing plasmalogen) described in the present specification, or a material in which it is optionally dissolved, dispersed or suspended in a solvent may be used as plasmalogen (or a composition containing plasmalogen) described in the present specification. Or else, a material obtained by subjecting a material to which a solvent has been added to a further treatment such as concentration, purification, etc., may be used as plasmalogen (or a composition containing plasmalogen) described in the present specification.

The concentration herein is not particularly limited, and may be carried out in an open system or may be carried out in a closed system, and it is preferable to carry out in a closed system. This concentration can be carried out by, for example, concentration by reduced pressure, concentration by heating, concentration by freezing, concentration using a membrane, etc., and among these, concentration by reduced pressure is preferable. Also, this concentration is preferably carried out under bubbling with an inert gas such as nitrogen, argon, etc., in order to prevent oxidation. When concentration by reduced pressure is carried out, it can be carried out, for example, at a temperature of about 10 to about 55° C., preferably about 25 to about 50° C., and more preferably about 40 to about 50° C. and, for example, for about 1 to about 72 hours, preferably about 6 to about 48 hours, and more preferably about 12 to about 36 hours. As a degree of pressure reduction, there may be mentioned, for example, about 60 mmHg.

The purification herein may be mentioned, which is not limited to these, for example, solvent extraction; reprecipitation; and chromatography such as thin layer chromatography, column chromatography, etc.

A desired additive(s) may be added, if necessary, during the treatment of the above-mentioned (A) or (B) or a treatment other than these, or to the obtained composition containing plasmalogen. An object of adding the additive(s) is not particularly limited, and, for example, there may be mentioned to add an antioxidant for the purpose of preventing oxidation of the plasmalogen, to add a preservative for the purpose of preserving the composition containing plasmalogen, to add a dispersant for the purpose of homogenization of the plasmalogen, and the like. As the additives which can be used, which is not limited to these, for example, an antioxidant such as ascorbic acid, tocopherol, erythorbic acid, sodium sulfite, dibutylhydroxytoluene, butylhydroxyanisole, catechin, etc.; a preservative such as sorbic acid, potassium sorbate, calcium sorbate, benzoic acid, sodium benzoate, propionic acid, sodium propionate, calcium propionate, sodium dehydro acetate, natamycin, pimalysin, polylysine, nisin, isopropyl paraoxybenzoate, isopropyl parahydroxybenzoate, isopropylparaben, etc.; a dispersant such as polysorbates, sodium lauryl sulfate, polyoxyethylene hydrogenated castor oil, lecithin, etc.; and the like, and these may be used alone or may be used in combination of two or more kinds.

The plasmalogen (or a composition containing plasmalogen) described in the present specification may enhance a verbal memorization ability and/or a visual memorization ability of a subject suffering from a disease that may affect a verbal memorization ability and/or a visual memorization ability such as a disease associated with central nervous system inflammation (which is not limited to these, for example, Alzheimer's disease, Parkinson's disease, dementia, schizophrenia, depression, etc.), and the like, or may enhance a healthy verbal memorization ability and/or visual memorization ability of a subject not suffering from these diseases, and preferably enhances a healthy verbal memorization ability and/or visual memorization ability of a subject not suffering from these diseases. Therefore, in one embodiment of the present invention, the plasmalogen described in the present specification (or a composition containing plasmalogen) enhances a healthy verbal memorization ability and/or visual memorization ability. In addition, in a preferred embodiment of the present invention, the plasmalogen described in the present specification (or a composition containing plasmalogen) enhances a healthy verbal memorization ability.

The term “healthy” to be used in the present invention means that the subject is not suffered from the above-mentioned diseases that may affect a verbal memorization ability and/or a visual memorization ability. The term “healthy” used in the present invention also includes the case where the verbal memorization ability and/or the visual memorization ability is/are lowered due to aging as compared with the young age. Whether or not a subject has a “healthy” verbal memorization ability and/or visual memorization ability may be judged, for example, the Mini-Mental State Examination (MMSE), and the like. When the Mini-Mental State Examination is used, for example, a subject with 20 points or more, preferably 22 points or more, more preferably 24 points or more, and further preferably 27 points or more out of 30 points may be judged as a subject having a healthy verbal memorization ability and/or visual memorization ability.

The “subject” used in the present invention is not limited to these, and there may be mentioned, for example, mammals including rodents such as mice, rats, hamsters, guinea pigs, etc.; Lagomorphs such as rabbits, etc.; Ungulates such as pigs, cows, goats, horses, sheep, etc.; Carnivora such as dogs, cats, etc.; Primates such as humans, monkeys, rhesus monkey, crab-eating monkey, marmosets, orangutans, chimpanzees, etc.; and the like, preferably Primates, and more preferably humans.

An intake of the composition containing plasmalogen is not particularly limited as long as the effect of enhancing a verbal memorization ability and/or a visual memorization ability is exhibited and, for example, as the plasmalogen, it is preferably about 1 to about 1,000 μg/kg body weight per day, more preferably about 10 to about 100 μg/kg body weight per day, further preferably about 12 to about 25 μg/kg body weight per day, and even more preferably about 15 to about 24 μg/kg body weight per day. In another embodiment of the present invention, the intake of the composition containing plasmalogen is, for example, as the plasmalogen, to a subject, preferably about 0.01 to about 100 mg per day, more preferably about 0.1 to about 10 mg per day, further preferably about 0.5 to about 5 mg per day, and even more preferably about 1 mg per day.

Accordingly, in one embodiment of the present invention, it is provided a composition described in the present specification, characterized by administering about 12 to about 25 μg/kg body weight per day of the plasmalogen described in the present specification.

In another embodiment of the present invention, it is provided a method for enhancing a verbal memorization ability and/or a visual memorization ability described in the present specification, which comprises administering about 12 to about 25 μg/kg body weight per day of the plasmalogen described in the present specification.

In another embodiment of the present invention, it is provided plasmalogen for enhancing a verbal memorization ability and/or a visual memorization ability described in the present specification, characterized by administering about 12 to about 25 μg/kg body weight per day of the plasmalogen described in the present specification.

The number of times of intake of the plasmalogen (or a composition containing plasmalogen) per day is not particularly limited as long as the effect of enhancing a verbal memorization ability and/or a visual memorization ability is exhibited and, for example, it may take once a day, or may take multiple times a day, and preferably once a day.

In one embodiment of the present invention, the composition containing plasmalogen described in the present specification is a food composition or a pharmaceutical composition. In a preferred embodiment of the present invention, the composition containing plasmalogen described in the present specification is a food composition. In a more preferred embodiment of the present invention, the food composition is a food for specified health uses or a food with functional use, or a health food other than these.

The composition containing plasmalogen described in the present specification may contain, which is not limited to these, for example, an excipient, a binder, a disintegrating agent, a lubricant, a sweetening agent, a coloring agent, a surfactant, a solubilizing agent, a dissolution assisting agent, a preservative, a pH adjusting agent, a suspending agent, an isotonizing agent, a buffer, an analgesic agent, an antioxidant, etc.

As the excipient, which is not limited to these, there may be mentioned, for example, lactose hydrate, white sugar, glucose, starch, sucrose, crystalline cellulose, mannitol, etc., and these may be used alone or may be used in combination of two or more kinds.

As the binder, which is not limited to these, there may be mentioned, for example, gum Arabic, hydroxypropyl methylcellulose, hydroxypropyl cellulose, hydroxyethyl cellulose, etc., and these may be used alone or may be used in combination of two or more kinds.

As the disintegrating agent, which is not limited to these, there may be mentioned, for example, corn starch, potato starch, carmellose calcium, carmellose sodium, low-substitution degree hydroxypropyl cellulose, croscarmellose sodium, crospovidone, carboxymethyl starch sodium, etc., and these may be used alone or may be used in combination of two or more kinds.

As the lubricant, which is not limited to these, there may be mentioned, for example, light anhydrous silicic acid, stearic acid, magnesium stearate, calcium stearate, sucrose fatty acid ester, polyethylene glycol, talc, etc., and these may be used alone or may be used in combination of two or more kinds.

As the sweetening agent, which is not limited to these, there may be mentioned, for example, sucrose, fructose, xylitol, sorbitol, aspartame, acesulfame potassium, sucralose, etc.

As the coloring agent, which is not limited to these, there may be mentioned, for example, yellow ferric oxide, black iron oxide, food yellow No. 4, food red No. 3, tar pigments, caramel, cacao pigments, titanium oxide, riboflavins, etc., and these may be used alone or may be used in combination of two or more kinds.

As the surfactant, which is not limited to these, there may be mentioned, for example, polysorbates, sodium lauryl sulfate, polyoxyethylene hydrogenated castor oil, etc., and these may be used alone or may be used in combination of two or more kinds.

As the solubilizing agent, which is not limited to these, there may be mentioned, for example, ethanol, propylene glycol, polyethylene glycol, sorbitan sesquioleate, sorbitan laurate, sorbitan palmitate, glyceryl oleate, glyceryl myristate, polyoxyethylene lauryl ether, polyoxyethylene nonylphenyl ether, glycerin, etc., and these may be used alone or may be used in combination of two or more kinds.

As the dissolution assisting agent, which is not limited to these, there may be mentioned, for example, polyethylene glycol; propylene glycol; cyclodextrin; sugar alcohol such as mannitol; benzyl benzoate; tris-aminomethane; cholesterol; triethanolamine; sodium carbonate; sodium citrate; an alcohol such as methanol, ethanol, propanol, isopropanol, etc.; single fatty acid such as acetic acid, propanoic acid, butanoic acid, pentanoic acid, hexanoic acid, heptanoic acid, myristic acid, stearic acid, oleic acid, etc., or an ester thereof; vegetable oil such as sesame oil, peanut oil, coconut oil, palm oil, soybean oil, olive oil, coconut oil, corn oil, cottonseed oil, castor oil, rapeseed oil, sunflower oil, etc.; and the like, and these may be used alone or may be used in combination of two or more kinds.

As the preservative, which is not limited to these, there may be mentioned, for example, sorbic acid, potassium sorbate, calcium sorbate, benzoic acid, sodium benzoate, propionic acid, sodium propionate, calcium propionate, sodium dehydroacetate, natamycin, pimalysin, polylysine, nisin, isopropyl paraoxybenzoate, isopropyl parahydroxybenzoate, isopropylparaben, etc., and these may be used alone or may be used in combination of two or more kinds.

As the pH adjusting agent, which is not limited to these, there may be mentioned, for example, acetic acid, lactic acid, tartaric acid, oxalic acid, glycolic acid, malic acid, citric acid, succinic acid, fumaric acid, phosphoric acid, hydrochloric acid, sulfuric acid, nitric acid, and a salt thereof, sodium hydroxide, potassium hydroxide, sodium hydrogen carbonate, potassium carbonate, etc., and these may be used alone or may be used in combination of two or more kinds.

As the suspending agent, which is not limited to these, there may be mentioned, for example, stearyl triethanolamine, sodium lauryl sulfate, laurylaminopropionic acid, lecithin, benzalkonium chloride, benzethonium chloride, glyceryl monostearate, polyvinyl alcohol, polyvinylpyrrolidone, sodium carboxymethyl cellulose, methyl cellulose, hydroxymethyl cellulose, hydroxyethyl cellulose, hydroxypropyl cellulose, etc., and these may be used alone or may be used in combination of two or more kinds.

As the isotonizing agent, which is not limited to these, there may be mentioned, for example, sodium chloride, glycerin, mannitol, etc., and these may be used alone or may be used in combination of two or more kinds.

As the buffer, which is not limited to these, there may be mentioned, for example, phosphates, acetates, carbonates, citrates, and a buffer containing these, etc., and these may be used alone or may be used in combination of two or more kinds.

As the analgesic agent, which is not limited to these, there may be mentioned, for example, benzyl alcohol, etc.

As the antioxidant, which is not limited to these, there may be mentioned, for example, ascorbic acid, tocopherol, erythorbic acid, sodium sulfite, dibutylhydroxytoluene, butylhydroxyanisole, catechin, etc., and these may be used alone or may be used in combination of two or more kinds.

In addition, the composition containing plasmalogen described in the present specification may contain not only plasmalogen but also other beneficial components (for example, other components that are beneficial for health or treatment) depending on the purpose thereof.

The composition containing plasmalogen described in the present specification may be formulated into a formulation by a known method itself, for example, such as tablets, coated tablets, powders, granules, fine granules, hard capsules, soft capsules, pills, liquids, suspensions, emulsions, jelly, chewable tablets, soft tablets, etc. These may be oral preparations or parenteral preparations but are preferably oral preparations. In the case of an oral preparation, in order to prevent decomposition of plasmalogen due to gastric acid or the like, it is preferable to process the preparation into an enteric one. As the enteric preparation, which is not limited to these, there may be mentioned, for example, an enteric tablet, an enteric hard capsule and an enteric soft capsule, and preferably an enteric soft capsule.

In the case of the soft capsules, hard capsules, or the like, as contents of the capsules, in addition to the above-mentioned additives, etc., for example, a base, etc., may be added.

As the base, which is not limited to these, there may be mentioned, for example, as an aqueous base such as water; an oily base such as medium chain fatty acid triglyceride, tricaprylin, caproic acid, capric acid, oleic acid, linoleic acid, linolenic acid, coconut oil, olive oil, rapeseed oil, peanut oil, corn oil, soybean oil, cottonseed oil, grape oil, safflower oil, etc.; and the like, and these may be used alone or may be used in combination of two or more kinds. Among these, oil bases are preferable, and medium chain fatty acid triglycerides are particularly preferable.

Further, in the case of soft capsules, a coating film covering the contents can be used. To the coating film, in addition to the above-mentioned additives, etc., for example, a coating base, a plasticizer, etc., may be added.

As the coating base, which is not limited to these, there may be mentioned, for example, gelatin, succinylated gelatin, starch (including modified starch), pullulan, polyvinyl alcohol copolymer, macrogol, carrageenan, glycerin, etc., and these may be used alone or may be used in combination of two or more kinds.

As the plasticizer, which is not limited to these, there may be mentioned, for example, sugar alcohols such as sorbitol, mannitol, etc.; and glycerin, etc., and these may be used alone or may be used in combination of two or more kinds.

When the composition containing plasmalogen described in the present specification is a hard capsule, it can be produced, for example, by mixing plasmalogen, an excipient, etc., to obtain capsule contents, and filling the capsule contents in the state of a solid, liquid, paste or the like in a gelatin capsule, etc.

When the composition containing plasmalogen described in the present specification is a soft capsule, it can be produced, for example, by filling the capsule contents into a coating film using a punching method using a rotary die type capsule molding device, a plating method in which the capsule contents are charged between two sheets of gelatin sheets, compressed from both sides by a mold and punching out therefrom, or a dropping method using a double nozzle, or the like, and molding and drying the same.

The shape of the soft capsule is not limited to these, and there may be mentioned, for example, oval type, oblong type, round type, long eggplant type, triangular type, rhombus, fish type, tube type, bottle type, and the like.

A content of plasmalogen in the composition containing plasmalogen described in the present specification or a material formulating the same is not particularly limited as long as an enhancement action of a verbal memorization ability and/or a visual memorization ability is/are exhibited and, for example, based on the total amount of the composition containing plasmalogen described in the present specification or a material formulating the same, for example, it is preferably made 0.05 to 99.9% by mass, more preferably made 0.1 to 80% by mass, and even more preferably made 0.15 to 40% by mass.

### EXAMPLES

In the following, the present invention will be explained in more detail by referring to Examples, but these Examples do not limit the scope of the present invention in any way.

### Preparation Example 1

### Preparation of Composition Containing Plasmalogen

The ascidian with shells was divided into two equal parts and dried in a cool air dryer by applying wind at about 45° C. for about 24 hours (the temperature in the cool air dryer becomes about 45° C.). To the dried material was added a mixed solution of ethanol and water (95% by volume:5% by volume), and the mixture was extracted under stirring at about 40° C. for about 2 hours. The solid and liquid were separated into a solid phase 1 and a liquid phase 1 with a stainless strainer (200 mesh). The liquid phase 1 was subjected to suction filtration to remove the solid material to obtain a liquid phase 2. To the solid phase 1 was added a mixed solution of ethanol and water (95% by volume:5% by volume), and after immersion at room temperature for about 10 hours, the solid and liquid were separated into a solid phase 2 and a liquid phase 3 with a stainless strainer (200 mesh). The liquid phase 3 was subjected to suction filtration to remove the solid material to obtain a liquid phase 4. The liquid phase 2 and the liquid phase 4 were combined and concentrated under reduced pressure at about 45° C. for about 24 hours. The concentrated product was stored at about 5° C. for about 8 days under nitrogen bubbling in a sealed state. Thereafter, water was added thereto to dilute the product, and the resulting material was allowed to stand at about 4° C. for about 3 days. Thereafter, the supernatant was removed by decantation treatment at about 4° C. to recover a liquid phase 5, and the content of the plasmalogen in the liquid phase 5 was measured by high performance liquid chromatography. Medium chain fatty acid triglyceride (available from The Nisshin OilliO group, Ltd.) and vitamin E (available from RIKEN VITAMIN CO., LTD.) were added to the liquid phase 5 so that the content of plasmalogen became 1% by mass to obtain a composition containing plasmalogen.

### Example 1

### Preparation of Plasmalogen-Containing Enteric Soft Capsule

100 mg of the composition containing plasmalogen obtained in Preparation Example 1 and 100 mg of medium chain fatty acid triglyceride (available from The Nisshin OilliO group, Ltd.) were filled into a coating film (32 parts by mass of glycerin (available from Sakamoto Yakuhin Kogyo Co., Ltd.), 26 parts by mass of gelatin (available from Nitta Gelatin Inc.), 26 parts by mass of modified starch (available from National Starch Food Innovation), 14 parts by mass of carrageenan (available from CP Kelco), and 2 parts by mass of cacao pigment (available from Mitsubishi-Chemical Foods Corporation)) using an existing rotary die-type capsule molding device to obtain an enteric soft capsule containing plasmalogen (the content of plasmalogen in the soft capsule: 1 mg).

### Comparative Example 1

### Preparation of Placebo Enteric Soft Capsule

Completely the same method as in Example 1 was repeated except for changing 100 mg of the composition containing plasmalogen and 100 mg of medium chain fatty acid triglyceride (available from The Nisshin OilliO group, Ltd.) in Example 1 to 200 mg of medium chain fatty acid triglyceride (available from The Nisshin OilliO group, Ltd.) to obtain a placebo enteric soft capsule.

### Test Example 1

In order to verify the memorization ability enhancement effect of plasmalogen, a human clinical test (randomized placebo-controlled, double-blind, parallel-group comparative test) by the method shown below was carried out.

Healthy adult men and women were made the subjects, and the subjects were randomly assigned to two groups of a plasmalogen intake group (n=25) and a control group (n=24). The background of the subjects is shown in Table 1. The plasmalogen intake group was ingested with one capsule of the enteric soft capsule containing plasmalogen prepared in Example 1, and the control group was ingested with one capsule of the placebo enteric soft capsule prepared in Comparative Example 1 once a day at an arbitrary timing (Intake period is 12 weeks).

Incidentally, for the subjects, during the test period, intake of foods for specified health uses, foods with functional use, and other foods or beverages with possible functionality was restricted, and use of equipment, devices, etc., that can contribute to improving brain function was restricted. In addition, from the date of obtaining the consent form for the test until the final test (test 12 weeks after ingestion) was completed, for the subjects, overdrinking and overeating were restricted and changing their lifestyle habits was prohibited.

Before taking the capsule and after the completion of taking the capsule for 4, 8, and 12 weeks, cognitive function in each subject was examined using Cognitrax (manufactured by Health Solution Co., Ltd.). Incidentally, Cognitrax is a system to evaluate cognitive function by carrying out 10 kinds of tests on the web using a computer. In this test, an amount of change was calculated by the following equation for each subject for the scores obtained with regard to the verbal memorization ability, the visual memorization ability, and the total memorization ability.

Amount of change in score=(Score after completion of intake of capsules for 4, 8, or 12 weeks)−(Score before intake of capsules)

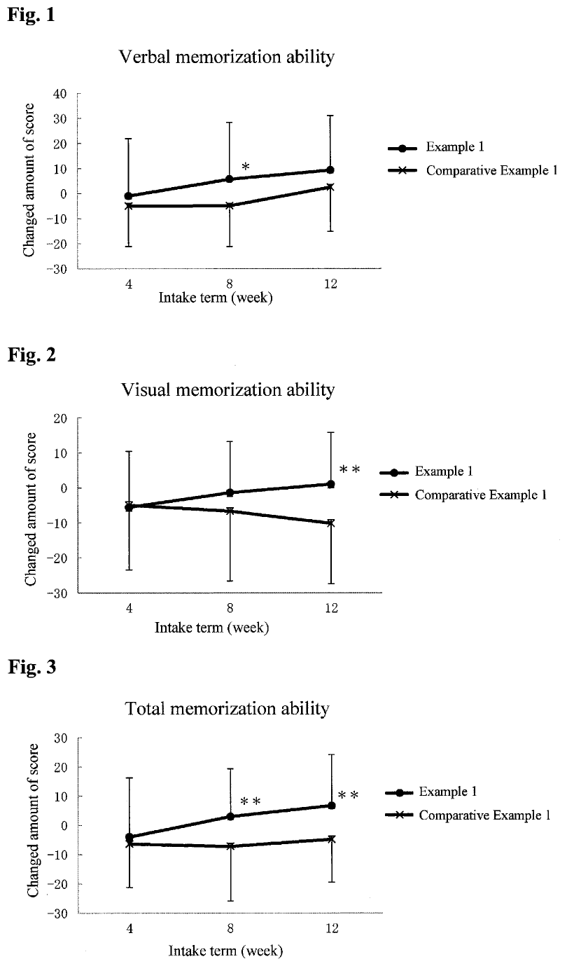

For the analysis of the obtained results, Student's t-test was used (used software: SPSS (version 23.0 or 24.0, manufactured by IBM Japan, Ltd.), and Microsoft Excel 2013 (manufactured by Microsoft)). The results are shown in FIG. 1 to FIG. 3.

TABLE 1

| | Plasmalogen | Control |
|---|---|---|
| | intake group | group |
| Average age ± standard | 45.6 ± 11.1 | 46.4 ± 10.8 |
| deviation (age) | | |
| Average body weight ± | 54.6 ± 11.9 | 59.5 ± 13.7 |
| standard deviation (kg) | | |
| Minimum body weight (kg) | 40.6 | 39.2 |
| Maximum body weight (kg) | 79.0 | 96.1 |

*The body weight shows data before administration. Incidentally, there is no significant change in body weight before and after administration.

From the results of Test Example 1 using Example 1 and Comparative Example 1, in the subject who ingested the enteric soft capsule containing plasmalogen, it can be seen that the verbal memorization ability, the visual memorization ability, and the total memorization ability were significantly enhanced.

Accordingly, plasmalogen has a function of enhancing the verbal memorization ability, the visual memorization ability, and the total memorization ability.

### Test Example 2

Plasmalogens comprised in the composition containing plasmalogen prepared in Preparation Example 1, sea squirt (produced in Miyagi prefecture) and scallop (produced in Miyagi prefecture) were subjected to LCMSMS analysis under the conditions described later, and the ratio of ethanolamine plasmalogen to choline plasmalogen comprised in the plasmalogen was analyzed.

A crushed product obtained by crushing sea squirt or scallop (about 100 g) into about 5 mm squares was freeze-dried to obtain about 15 g of dried product. To the product was added 95% ethanol, and the mixture was extracted by ultrasonication at about 40° C. for about 2 hours. The solid and liquid were separated into a solid phase and a liquid phase by a suction funnel, and 95% ethanol (about 100 g) was added to the solid phase to separate the liquid phase. The liquid phases were combined, 1.3-fold amount (in terms of weight) of water based on the liquid phase was added, and the mixture was allowed to stand at about 4° C. for about 3 days. The upper layer was removed by decantation treatment and centrifugation (4,000 rpm, 5 minutes). The precipitates at the lower layer were dissolved in ethanol, which was made a sample.

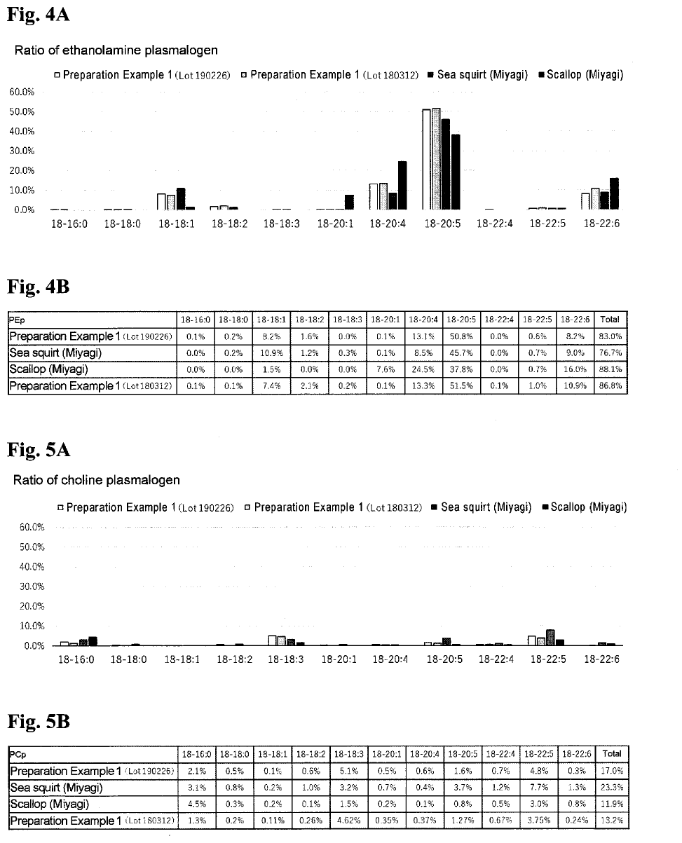

Incidentally, the ratio (mass ratio) of ethanolamine plasmalogen and choline plasmalogen was calculated so that the total of these became 100%. The results are shown in FIG. 4A to FIG. 5B.

LCMSMS Analysis Conditions

### [Standard Product]

Ethanolamine plasmalogen (PEp) 18-18:1 (available from Merck)

Choline plasmalogen (PCp) 18-18:1 (available from Merck)

*18-18:1 means that the sn-1 position is C18 and the sn-2 position is a group derived from oleic acid (18:1).

### [Analytical Conditions]

Column: L-column 2 (2.1×150 mm, CERI)

Column temperature: 50° C.

Mobile phase A: acetonitrile/water (60/40) 10 mM ammonium formate, 0.1% formic acid

Mobile phase B: isopropanol/acetonitrile (90/10) 10 mM ammonium formate, 0.1% formic acid

Gradient: 0 to 27 minutes (A: 70%→A: 1%), 27 to 31 minutes (A: 1%), 31 to 31.1 minutes (A: 1%→A: 70%), 31.1 to 35 minutes (A: 70%)

Flow rate: 300 μL/min, Injection amount: 5 μL

Detector: 3200QTRAP (manufactured by AB Sciex LLC)

### [Measured Ion]

TABLE 2

| | Product ion | 18-18:1 |
|---|---|---|
| | (ion mode) | (precursor/product) |
| Ethanol plasmalogen | [R₂COO]⁻ | (728.6/281.4) |
| (PEp) | (negative ion mode) | |
| Choline plasmalogen | [H₂PO₄—(CH₂)₂—N(CH₃)₃]⁺ | (772.6/184.3) |
| (PCp) | (positive ion mode) | |

*As PEp and PCp, 11 species for each were detected in which the sn-1 position was C18, and the sn-2 position was of 16:0 (a group derived from palmitic acid), 18:0 (a group derived from stearic acid), 18:1 (a group derived from oleic acid), 18:2 (a group derived from octadecadienoic acid), 18:3 (a group derived from octadecatrienoic acid), 20:1 (a group derived from eicosenoic acid), 20:4 (a group derived from eicosatetraenoic acid), 20:5 (a group derived from eicosapentaenoic acid), 22:4 (a group derived from docosatetraenoic acid), 22:5 (a group derived from docosapentaenoic acid) or 22:6 (a group derived from docosahexaenoic acid). With regard to the molecular species for which standard products were not commercially available, the mass was calculated from the area value as PEp 18-18:1 or PCp 18-18:1 estimating from the retention time, precursor, and product ion.

From the results of Test Example 2, it can be understood that the plasmalogen comprised in the composition containing plasmalogen prepared in Preparation Example 1, sea squirt and scallop contains ethanolamine plasmalogen and choline plasmalogen. Further, it can be understood that the mass ratio of ethanolamine plasmalogen to choline plasmalogen comprised in the plasmalogen is about 7:about 3 to about 9 to about 1. Accordingly, it can be considered that the plasmalogen containing ethanolamine plasmalogen and choline plasmalogen (in particular, a material having a mass ratio of ethanolamine plasmalogen to choline plasmalogen of about 7:about 3 to about 9 to about 1) is useful for enhancing memorization power such as a verbal memorization ability, a visual memorization ability, a total memorization ability and the like.

As the ethanolamine plasmalogen, those having a group derived from oleic acid, a group derived from octadecadienoic acid, a group derived from eicosenoic acid, a group derived from eicosatetraenoic acid, a group derived from eicosapentaenoic acid, and a group derived from docosahexaenoic acid (in particular, a group derived from oleic acid, a group derived from eicosatetraenoic acid, a group derived from eicosapentaenoic acid, and a group derived from docosahexaenoic acid) at the sn-2 position were contained with a large amount. On the other hand, as the choline plasmalogen, those having a group derived from palmitic acid, a group derived from octadecatrienoic acid, a group derived from eicosapentaenoic acid, and a group derived from docosapentaenoic acid at the sn-2 position were contained with a large amount.

As shown by the results of the Examples hereinabove, according to the present invention, a composition containing plasmalogen for enhancing memorization power such as a verbal memorization ability, a visual memorization ability, a total memorization ability and the like can be provided. In addition, according to the present invention, it can be provided a method for enhancing a memorization power such as a verbal memorization ability, a visual memorization ability, a total memorization ability, and the like, which comprises administering an effective amount of plasmalogen to a subject in need thereof. Further, according to the present invention, it can be provided plasmalogen for enhancing a memorization power such as a verbal memorization ability, a visual memorization ability, a total memorization ability, and the like.

1.-14. (canceled)

15. A method for enhancing a verbal memorization ability and/or a visual memorization ability, comprising administering an effective amount of plasmalogen to a subject in need thereof.

16. The method according to claim 15, wherein the method is for enhancing a verbal memorization ability.

17. The method according to claim 15, wherein the method is for enhancing a verbal memorization ability and a visual memorization ability.

18. The method according to claim 15, wherein plasmalogen comprises ethanolamine plasmalogen and choline plasmalogen.

19. The method according to claim 18, wherein a mass ratio of ethanolamine plasmalogen to choline plasmalogen comprised in the plasmalogen is 7:3 to 9:1.

20. The method according to claim 15, wherein the plasmalogen is derived from a plasmalogen-containing animal tissue.

21. The method according to claim 20, wherein the animal tissue is derived from aquatic invertebrates.

22. The method according to claim 21, wherein the aquatic invertebrates are aquatic invertebrates of phylum Chordata, subphylum Urochordata, class Ascidiacea.

23. The method according to claim 22, wherein the aquatic invertebrates of the phylum Chordata, subphylum Urochordata, class Ascidiacea are aquatic invertebrates of the genus Halocynthia.

24. The method according to claim 23, wherein the aquatic invertebrates of the genus Halocynthia is ascidian or golden sea squirt.

25. The method according to claim 15, wherein the method is for enhancing a healthy verbal memorization ability.

26. The method according to claim 15, wherein 12 to 25 μg/kg body weight per day of the plasmalogen is administered.
